(19) United States
(12) Patent Application Publication
(10) Pub. No.: US 2024/0148855 A1
(43) Pub. Date: May 9, 2024

(54) CANCER VACCINE AND METHOD OF USE THEREOF

(71) Applicants: NE1 Inc., New York, NY (US); United States Department of Health and Human Services and the National Institute of Health, Bethesda, MD (US)

(72) Inventors: Zhengping ZHUANG, Bethesda, MD (US); Herui WANG, Mclean, VA (US); Juan YE, Bethesda, MD (US); Bin LU, Falls Church, VA (US)

(21) Appl. No.: 18/550,895

(22) PCT Filed: Mar. 14, 2022
(86) PCT No.: PCT/US2022/020165

§ 371 Date: Sep. 15, 2023

Related U.S. Application Data
(60) Provisional application No. 63/162,990, filed on Mar. 18, 2021.

(51) Int. Cl. A61K 39/145; A61K 39/00; A61K 39/215; A61K 39/245; A61K 45/06; C07K 16/28

(52) U.S. Cl. CPC A61K 39/145; A61K 39/215; A61K 39/245; A61K 45/06; C07K 16/2878; A61K 2039/505; A61K 2039/5152; A61K 2039/5156; A61K 2039/53; A61K 2039/54; A61K 2039/55555; A61K 2039/55583; A61K 2039/585; C12N 2710/16034; C12N 2760/16134; C12N 2770/20034

(57) ABSTRACT

The present disclosure generally relates to a personalized cancer vaccine having attenuated cancer cells transfected with at least one expression construct. The expression construct is capable of secretory expression of an antigenic polypeptide that could be derived from a protein from a virus. The personalized tumor vaccine, when administered to a subject in need thereof, is effective to activate an immune response.

### CROSS-REFERENCE TO RELATED APPLICATIONS

The present application claims the benefit of U.S. Provisional Patent Application No. 63/162,990, filed Mar. 18, 2021, the contents of which are incorporated into the present application in their entirety.

### REFERENCE TO GOVERNMENT GRANTS

This invention was made with government support under grant number ZIA BC011773-01 by the National Cancer Institute of the National Institutes of Health. The Government has certain rights in the invention.

### FIELD OF THE INVENTION

The present invention relates generally to the preparation and use of a cancer vaccine, more specifically, a personalized cancer vaccine, for cancer immunotherapy.

### BACKGROUND

The development and use of vaccines are some of the major success stories of modern medicine. Through vaccination, the immune system is harnessed to protect against diseases, mainly infectious diseases. Over the past several decades, vaccine technology has evolved and most recently, mRNA vaccines have emerged as a breakthrough platform and have been showing their game-changing power in the worldwide pandemic fight against a human coronavirus.

Unlike a normal vaccine, mRNA vaccines work by introducing an mRNA sequence coded for a disease-specific antigen directly into the body. Based on in situ protein expression, the antigen is produced within the body and can be instantly recognized by the immune system. mRNA vaccines have been shown to trigger a balanced immune response comprising both cellular and humoral immunity. See RNA Biol. 2012; 9(11): 1319-1330

Meanwhile, the fight against cancer has turned more and more attention to cancer immunotherapy that treats cancer patients with components of the immune system. One particular cancer immunotherapy involving so-called immune checkpoint inhibitors (ICIs), such as anti-PD1/PD-L1 and anti-CTLA-4, has gained a prominent current status for its superior outcome in treating various types of advanced cancers. Yet, over the last decades, cancer vaccines as well as adoptive T cell therapies, which are of cancer immunotherapy but more technically demanding, have also shown efficacy in clinical trials. See Rosenberg, S. A. Science Translational Medicine 2012; 4, 127-128.

Cancer vaccine development relies on identification of tumor-specific antigens called neoantigens that are generated due to the genetic instability of cancer cells. Neoantigens are highly immunogenic as they are not expressed in normal tissues. Also, because neoantigens vary highly depending on the individual tumor, the cancer vaccine thus developed is amenable for personalized treatment.

Clearly, to leverage mRNA vaccine technology to develop cancer vaccines presents a promising path forward. Indeed, the RNA neoantigen vaccine has currently been under intensive study. As more immunotherapeutic drugs are being explored, the development of mRNA new technology also calls for a novel design for a cancer vaccine.

The inventors address this need in the disclosure herein.

### SUMMARY OF THE INVENTION

When herd immunity, also known as “population immunity”, is achieved against an infectious disease, e.g., influenza flu or Covid-19, it is projected that a general population has developed immunity against a pathogenic antigen of that infectious disease either through vaccination or through previous infection. To take advantage of the herd immunity among a population, including cancer patients, this invention is made to trigger an immune response by targeting expression of such a herd-immunized antigen either in ex vivo cultured cancer cells or possibly tumors. The approach has a potential to expand the arsenal against the cancer disease.

One aspect of this invention relates to a personalized cancer vaccine containing attenuated cancer cells that express an antigenic polypeptide. The antigenic polypeptide is membrane anchored. Further, the personalized tumor vaccine, when administered to a subject in need thereof, is effective to activate an immune response.

In a personalized cancer vaccine, the expression of an antigenic polypeptide can be achieved through, for example, mRNA electroporation or a virus vector (including, but not limited to an adenovirus, or lentivirus). Also, the attenuated cancer cells can be prepared by the following steps: (1) harvesting cancer cells from a biopsy of a site of tumor from the individual, (2) culturing the harvested cancer cells to a therapeutically relevant amount, (3) modification of the cultured cells with mRNA or viruses to express the antigenic polypeptide, (4) irradiating the modified cultured cancer cells, and, (5) adding one or more adjuvants such as TLR agonists to establish vaccine.

In one embodiment, the antigenic polypeptide of the personalized cancer vaccine is derived from a protein from a strain of Influenza A virus or an immunogenic fragment thereof Examples of the protein from a strain of Influenza A virus could include influenza hemagglutinin 1 (HA1), HA2, HA7, HA10, neuraminidase protein, or a combination thereof.

In another embodiment, the antigenic polypeptide of the personalized cancer vaccine could be a herpes simplex virus antigenic polypeptide or an immunogenic fragment thereof.

In yet another embodiment, the antigenic polypeptide of the personalized cancer vaccine is derived from a protein from human coronavirus SARS-CoV-2 or an immunogenic fragment thereof. Examples of the protein from SARS-CoV-2 includes Coronavirus (CoV) S1 protein (spike protein), an envelope protein, a membrane protein, a nucleocapsid protein, or a combination thereof.

The personalized cancer vaccine can further contain a phagocytosis stimulating agent and an immunostimulatory adjuvant. The phagocytosis stimulating agent could be a mannan, and the immunostimulatory adjuvant could be a Toll like receptor (TLR) agonist, an anti-CD40 antibody, or a combination thereof. Examples of the TLR Agonists include LTA, Poly I:C, and R-848, and the anti-CD40 antibodies can be agonistic.

Another aspect of this invention relates to a cancer vaccine. The cancer vaccine includes a first agent comprising at least one ribonucleic acid (RNA) polynucleotide(s); and a second agent comprising a phagocytosis stimulating agent and an immunostimulatory adjuvant. Also, the cancer vaccine is formulated for delivery to a tumor organ. Upon being delivered to the tumor organ, the first agent is configured to produce a membrane anchored antigenic polypeptide therein and trigger an immune response; and the second agent is effective to amplify the immune response.

Specifically, the delivery of the cancer vaccine is through an intratumoral injection. Just as in the above-described personalized cancer vaccine, the membrane anchored antigenic polypeptide of the cancer vaccine can be derived from a strain of Influenza A virus, a herpes simplex virus, or human coronavirus SARS-CoV-2. The first agent of the cancer vaccine could be formulated in a lipid nanoparticle composition. As for the second agent, the phagocytosis stimulating agent, again, could be a mannan, and the immunostimulatory adjuvant, again, could be a Toll like receptor (TLR) agonist, an anti-CD40 antibody, or a combination thereof.

Still another aspect of this invention relates to another personalized cancer vaccine that includes a first agent of comprising at least one ribonucleic acid (RNA) polynucleotide and a second agent comprising a phagocytosis-stimulating agent and an immunostimulatory adjuvant. The personalized cancer vaccine is formulated to be administered to a cancer patient in need thereof and the RNA polynucleotide encodes at least one neoantigen derived from said cancer patient. Upon administration to the cancer patient, the first agent is configured to produce an antigenic polypeptide therein and trigger an immune response; and the second agent is effective to amplify the immune response.

In the above personalized cancer vaccine, the RNA polynucleotide could contain at least an open reading frame of one tandem minigene construct comprising a sequence encoding at least one neoantigen derived from said cancer patient.

In yet another aspect of the current invention, in the above personalized cancer vaccine, the first agent can be formulated in a nanoparticle composition, the phagocytosis stimulating agent can be a mannan, the immunostimulatory adjuvant can be a Toll like receptor (TLR) agonist, an anti-CD40 antibody, or a combination thereof.

The just-described cancer vaccine or personalized cancer vaccine confers therapeutical values. Consequently, yet another aspect of this invention relates to a method for treating a cancer by administering to a subject having the cancer an effective amount of an above-described cancer vaccine or personalized cancer vaccine. This cancer vaccine or personalized cancer vaccine can be administered by a subcutaneous inoculation.

The above methods of this invention can be applied to a mammal, e.g., a human or a mouse.

These and other embodiments and features of the disclosure will become more apparent through reference to the following description, the accompanying FIGS., and the claims. Furthermore, it is to be understood that the features of the various embodiments described herein are not mutually exclusive and can exist in various combinations and permutations.

### BRIEF DESCRIPTION OF THE DRAWINGS

FIG. 1 shows an illustration of the disclosed cancer vaccine strategy. Tumor tissues from cancer patients are dissociated into single cells and cultured. These cells are subject to introduction of foreign immunogenic polypeptides via gene delivery system (for example, mRNA, virus vectors, etc). Coronavirus (CoV) S protein (spike protein) is shown in the drawing as an example. Such modified tumor cells will be irradiated and administered to the same patient. Inside the human body with herd immunity to S protein: (1) S protein at the tumor cell surface will be recognized by specific antibodies and antibody-dependent cellular phagocytosis will occur; (2) T cells that recognize MHC1-epitope peptide of S protein will also attack irradiated tumor cells and cause tumor cell death. After the cancer cells break down and are phagocyted by antigen-presenting cells (APC), neoantigens from the derived tumor cells will be presented by APC, inducing tumor-specific adaptive immune response. To bolster APC functions, three TLR ligands (LTA, Poly (I:C) and R-848) and immunostimulatory anti-CD40-mAb are incorporated as immunological adjuvants. Activation of innate immune cells with TLR agonists generates chemokines and inflammatory cytokines that promote APC maturation and tumor antigen processing. CD40, a tumor necrosis factor receptor, is expressed on T helper cells and APCs. CD40 ligation with anti-CD40-mAb results in APC activation and induction of adaptive immunity.

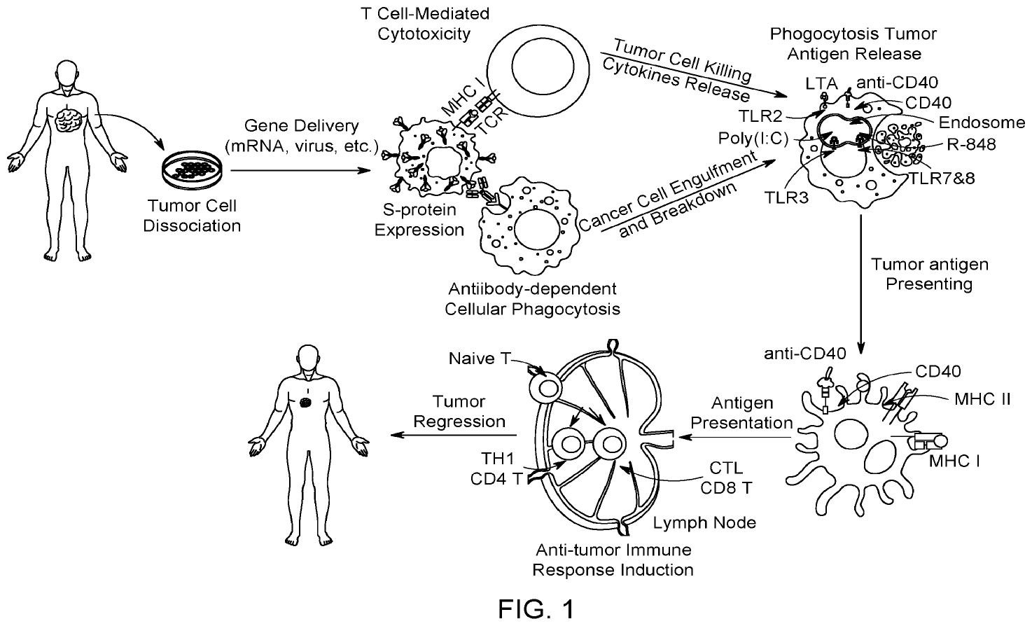

### DETAILED DESCRIPTION

Throughout this disclosure, various quantities, such as amounts, sizes, dimensions, proportions, and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

While immunotherapeutics, including checkpoint blockers, vaccines, and chimeric antigen receptor (CAR) T-cell therapies, are proliferating in the treatment of cancer, they have also identified some tumors that do not respond very well to the therapy. It is found that those tumors, dubbed as “immunologically cold,” are often with low T-cell infiltration. By contrast, their counterpart “immunologically hot” tumors contain high levels of infiltrating T cells and more antigens, making them more recognizable by the immune system and more likely to trigger a strong immune response. Hence, conversion of a “cold” to “hot” immunogenic phenotype is an important step to make successful immunotherapy.

A promising immunotherapeutic agent capable of converting an “immunologically cold” tumor to an “immunologically hot” tumor is a combination of mannan, a polysaccharide derived from Saccharomyces cerevisiae, TLR (Toll like receptor) ligands, and agonistic anti-CD40-monoclonal antibody (abbreviated as MBTA).

Mechanistically, MBTA acts by stimulating acute inflammatory responses in the tumor microenvironment and thereby enhancing tumor cell phagocytosis and inducing the development of tumor-specific adaptive immune responses. Specifically, tumor cell phagocytosis is facilitated by the linkage of mannan to biocompatible anchor for cell membrane (Mannan-BAM), which exploits mannan's recognition by pattern recognition receptors on innate immune cells. TLR ligands and anti-CD40-monoclonal antibody function as immunostimulatory adjuvants on antigen presenting cells (APCs).

This invention nevertheless focuses on two parallel approaches to advance immunotherapies relying on an antigen having established herd immunity in a general population.

One approach is to express such an antigen in ex vivo cultured cancer cells. Those cancer cells are derived from cancer patients so that they can be used as personalized cancer vaccines. See for example WO2021119376, the contents of which is incorporated by reference in its entirety. After being engineered to express the antigen having herd immunity, those cancer vaccine cells are expected to be readily recognized by the immune system, cascading to an eventual immunotherapy to rid similar cancer cells off the body.

Another novel approach that the inventors utilize in the current invention is the development of mRNA-based cancer vaccines so that such an antigen would be expressed in situ in target tumors. It is expected once the immune system is tricked to attack the target tumors, more tumor-specific antigens could be exposed to induce second or third wave of immune responses.

The present invention also includes preparing another personalized tumor vaccine by delivering an mRNA neoantigen vaccine. A common theme of all the above approaches is to co-apply MBTA, which has exhibited unexpectedly strong activities in enhancing immune responses against the cancer.

In practice, some of the described approaches could target primary tumors, whereas others could prove effective against distal, untreated tumors.

Before the present invention is described in more details, it is to be understood that this invention is not limited to a particular method described, as such may, of course, vary. It is also to be understood that the terminology used herein is for the purpose of describing particular embodiments only, and is not intended to be limiting, since the scope of the present invention will be limited only by the appended claims.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

The terms “cell,” and “cells,” and “cell population,” used interchangeably, intend one or more mammalian cells. The term includes progeny of a cell or cell population. Those skilled in the art will recognize that “cells” include progeny of a single cell, and there are variations between the progeny and its original parent cell due to natural, accidental, or deliberate mutation and/or change.

The terms “cell proliferation” and “to proliferate” as used herein refer to the amplification of the cell-by-cell division.

A “cancer cell” as used herein refers to a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell” or “cancerous cell,” and encompasses cancer cells of a solid tumor, a semi-solid tumor, a primary tumor, a metastatic tumor, and the like.

Immune effector cells are the transiently activated cells that defend the body in an immune response. Once the triggering antigen/pathogen has been cleared, immune effector cells eventually stop proliferating and die. Effector B cells are called plasma cells and secrete antibodies, and activated T cells include cytotoxic T cells and helper T cells.

“Immunotherapy” refers to treatment of disease (e.g., cancer) by modulating an immune response to a disease antigen. In the context of the present application, immunotherapy refers to providing an anti-cancer immune response in a subject by administration of an antibody (e.g., a monoclonal antibody) and/or by administration of an antigen that elicits an anti-tumor antigen immune response in the subject.

Those skilled in the art understand how to make and apply vaccines, including personalized cancer vaccines.

The terms “antigen” and “epitope” are well understood in the art and refer to the portion of a macromolecule (e.g., a polypeptide) which is specifically recognized by a component of the immune system, e.g., an antibody or a T-cell antigen receptor.

As used herein, the term “antigen” encompasses antigenic epitopes, e.g., fragments of an antigen which are antigenic epitopes. Epitopes can be recognized by antibodies in solution, e.g. free from other molecules. Epitopes can be recognized by T-cell antigen receptor when the epitope is associated with a class I or class II major histocompatibility complex molecule.

An “effective amount” is an amount sufficient to effect beneficial or desired clinical results. An effective amount can be administered in one or more administrations. For purposes of this invention, an effective amount of reagent antibodies is an amount that is sufficient to diagnose, palliate, ameliorate, stabilize, reverse, slow or delay the progression of the disease state.

“Immunotherapy” as used herein refers to treatment of a disease (e.g., cancer) by modulating an immune response. In the context of the present application, immunotherapy refers to providing an anti-cancer immune response in a subject by administration of a personalized tumor vaccine that elicits an anti-tumor immune response in the subject.

A “TLR” refers to a toll-like receptor of any species origin, e.g., human and rodent. Examples of TLR thereof include TLR1, TLR2, TLR3, TLR4, TLR5, TLR6, TLR7, TLR8, TLR9, TLR 10 and TLR11.

A “TLR agonist” refers to a molecule that acts as an agonist of at least one TLR.

A “CD40 agonist” herein refers to a molecule that functions as a CD40 agonist signal such as a CD40 agonistic antibody (e.g., an anti-CD40 monoclonal antibody). It can also refer to a CD40L polypeptide, fragment, or conjugate thereof. In general, ligands that bind CD40 may act as a CD40 agonist. Also, CD40 agonists according to the invention may include aptamers that bind CD40.

The term “solid tumor” or “solid cancer” as used herein refers to tumors that usually do not contain cysts or liquid areas. Solid tumors can include brain and other central nervous system tumors (including but not limited to tumors of the meninges, brain, spinal cord, cranial nerves and other parts of central nervous system, e.g. glioblastomas or medulla blastomas); head and/or neck cancer; breast tumors; circulatory system tumors (including but not limited to heart, mediastinum and pleura, and other intrathoracic organs, vascular tumors and tumor-associated vascular tissue); excretory system tumors (including but not limited to tumors of kidney, renal pelvis, ureter, bladder, other and unspecified urinary organs); gastrointestinal tract tumors (including but not limited to tumors of esophagus, stomach, small intestine, colon, colorectal, rectosigmoid junction, rectum, anus and anal canal, tumors involving the liver and intrahepatic bile ducts, gall bladder, other and unspecified parts of biliary tract, pancreas, other and digestive organs); oral cavity tumors (including but not limited to tumors of lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (including but not limited to tumors of vulva, vagina, Cervix uteri, Corpus uteri, uterus, ovary, and other sites associated with female genital organs, placenta, penis, prostate, testis, and other sites associated with male genital organs); respiratory tract tumors (including but not limited to tumors of nasal cavity and middle ear, accessory sinuses, larynx, trachea, bronchus and lung, e.g. small cell lung cancer or non-small cell lung cancer); skeletal system tumors (including but not limited to tumors of bone and articular cartilage of limbs, bone articular cartilage and other sites); skin tumors (including but not limited to malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

The term “liquid cancer” or “liquid tumor” as used herein refers to cancer cells that are present in body fluids, such as blood, lymph and bone marrow. Liquid cancers include leukemia, myeloma, myelodysplastic syndrome (MDS), and liquid lymphomas. Liquid lymphomas include lymphomas that contain cysts or liquid areas. Liquid cancers as used herein do not include solid tumors, such as sarcomas and carcinomas or solid lymphomas that do not contain cysts or liquid areas.

An “attenuated” cell as used herein refers to a cell that is alive but replication deficient. The attenuated cell may be alive but unable to complete its cell-cycle. The attenuated cell may have a limited capacity to replicate, express proteins, and to develop through some life cycle stages, for example, an attenuated cell may be arrested at a particular life cycle stage and is unable to developmentally progress beyond that stage.

The term “attenuated cancer cell” as used herein refers to a cancer cell that is attenuated and with reduced oncogenicity. The attenuated cancer cell may be unable to cause or give rise to a tumor. The attenuated cancer cell may also be unable to metastasize or increase a tumor burden of a subject with the tumor. The attenuated cancer cell may comprise damages in their DNA. The attenuated cancer cell can be obtained by various means, for example, by physical and chemical treatments. The attenuated cancer cell can be obtained by irradiation treatments.

The term “personalized tumor vaccine” as used herein refers to a tumor vaccine that can direct a subject-specific immune response to a tumor of a subject. Such a response may be specific to a specific type of tumor from a specific subject. The personalized tumor vaccine as used herein comprises attenuated cancer cells. The personalized tumor vaccine may elicit an adaptive immune response to a tumor or tumor cells of a subject.

The immunotherapeutic strategy, introduced in this invention, leverages the use of phagocytosis stimulating agents and immunostimulatory adjuvants for directing an immune response against a subject-specific cancer or cancer cells. The personalized tumor vaccine described here having immunotherapeutic potential delivered as part of a whole attenuated cancer cell vaccine generates a potent adaptive immune response capable of preventing or controlling tumor growth and inducing tumor regression in a subject-specific subset of primary or metastatic tumors. Personalized tumor vaccines are used to train the immune system to find and destroy, attack, kill, or inhibit subject-specific cancer cells.

In some instances, a personalized tumor vaccine can direct the immune system of a subject to target subject-specific cancer cells. In some cases, a personalized tumor vaccine can direct the immune system of a subject to discriminate subject-specific cancer cells from other cancer cells not of the same subject. In some cases, a personalized tumor vaccine can direct the immune system of a subject to discriminate subject-specific cancer cells from cancer cells of other subjects. Such vaccine can make use of a phagocytosis stimulating agent or agents specific to the subject-specific cancer cells. A phagocytosis stimulating agent or agents can allow the immune system to discriminate the subject-specific cancer cells from the non-cancer cells, cancer cells from other tumors, or cancer cells from other subjects. When an immune system is presented with a phagocytosis stimulating agent or agents specific of a subject-specific cancer, the immune system will be activated to target the cancer cells sharing the same subject-specificity, even if a cancer is not present. Such activation of the immune system will create long lasting subject-specific immune memory. When the immune system encounters a cancer with the same or similar subject-specificity in the future, it will immediately activate a subject-specific immune response against the cancer cell.

A personalized tumor vaccine can be a prophylactic or preventative vaccine. A personalized tumor vaccine can be a therapeutic or treatment vaccine. In some embodiments, a personalized tumor vaccine can be administered to a subject in need thereof before the subject is diagnosed with a cancer. In some embodiments, a personalized tumor vaccine can be administered to a subject in need thereof after the subject is diagnosed with a cancer.

A personalized tumor vaccine can comprise a phagocytosis stimulating agent or derivative herein and thereof; an immunostimulatory adjuvant or derivative herein and thereof, and an attenuated cancer cell, cell population or derivative herein and thereof.

In some instances, a personalized tumor vaccine can comprise a phagocytosis stimulating agent or derivative herein and thereof; an immunostimulatory adjuvant or derivative herein and thereof, and from about 1.0×10∧3 to about 1.0×10∧7 attenuated cancer cells. In some instances, a personalized tumor vaccine can comprise a mannan; an immunostimulatory adjuvant or derivative herein and thereof, and an attenuated cancer cell, cell population or derivative herein and thereof. In some instances, a personalized tumor vaccine can comprise about 2 mg/dose to about 200 mg/dose mannan; an immunostimulatory adjuvant or derivative herein and thereof, and an attenuated cancer cell, cell population or derivative herein and thereof. In some cases, a personalized tumor vaccine can comprise a phagocytosis stimulating agent or derivative herein and thereof conjugated to biocompatible anchor for cell membrane (BAM); an immunostimulatory adjuvant or derivative herein and thereof, and an attenuated cancer cell, cell population or derivative herein and thereof. In some cases, a personalized tumor vaccine can comprise a mannan conjugated to BAM an immunostimulatory adjuvant or derivative herein and thereof, and an attenuated cancer cell, cell population or derivative herein and thereof. In some cases, a personalized tumor vaccine can comprise a mannan conjugated to from about 0.2 mg/dose to about 20 mg/dose BAM; an immunostimulatory adjuvant or derivative herein and thereof, and an attenuated cancer cell, cell population or derivative herein and thereof.

In some instances, a personalized tumor vaccine can comprise a phagocytosis stimulating agent or derivative herein and thereof; a Toll like receptor (TLR) agonist; and an attenuated cancer cell, cell population or derivative herein and thereof. In some cases, a personalized tumor vaccine can comprise a phagocytosis stimulating agent or derivative herein and thereof; R-848, poly (I:C), lipoteichoic acid (LTA), or combinations thereof; and an attenuated cancer cell, cell population or derivative herein and thereof. In some cases, a personalized tumor vaccine can comprise a phagocytosis stimulating agent or derivative herein and thereof; from about 0.05 mg/dose to about 5 mg/dose R-848, poly (I:C), lipoteichoic acid (LTA), or combinations thereof; and an attenuated cancer cell, cell population or derivative herein and thereof. In some cases, a personalized tumor vaccine can comprise a phagocytosis stimulating agent or derivative herein and thereof; R-848, from about 0.05 mg/dose to about 5 mg/dose poly (I:C), lipoteichoic acid (LTA), or combinations thereof; and an attenuated cancer cell, cell population or derivative herein and thereof. In some cases, a personalized tumor vaccine can comprise a phagocytosis stimulating agent or derivative herein and thereof; R-848, poly (I:C), from about 0.05 mg/dose to about 5 mg/dose lipoteichoic acid (LTA), or combinations thereof; and an attenuated cancer cell, cell population or derivative herein and thereof.

In some instances, a personalized tumor vaccine can comprise a phagocytosis stimulating agent or derivative herein and thereof; an anti-CD40 antibody; and an attenuated cancer cell, cell population or derivative herein and thereof. In some instances, a personalized tumor vaccine can comprise a phagocytosis stimulating agent or derivative herein and thereof; from about 0.04 mg/dose to about 4 mg/dose anti-CD40 antibody; and an attenuated cancer cell, cell population or derivative herein and thereof.

In some instances, a personalized tumor vaccine can comprise a mannan attached to BAM, a R-848, a poly (I:C), an LTA, an anti-CD40 antibody, and irradiated cancer cells. In some instances, a personalized tumor vaccine can comprise from about 0.05 mg/dose to about 5 mg/dose mannan attached to from about 0.05 mg/dose to about 5 mg/dose BAM, from about 0.05 mg/dose to about 5 mg/dose R-848, from about 0.05 mg/dose to about 5 mg/dose poly (I:C), from about 0.05 mg/dose to about 5 mg/dose LTA, from about 0.04 mg/dose to about 4 mg/dose anti-CD40 antibody, and from about 1.0×10∧3 to about 1.0×10∧7 irradiated cancer cells. Phagocytosis stimulating agents.

A personalized tumor vaccine described herein and thereof can comprise one phagocytosis stimulating agent. In some instances, a phagocytosis stimulating agent can comprise a nucleic acid, amino acid, nucleotide, carbohydrate, lipid, small molecule, ion, compound, any derivatives herein and thereof, or any combinations herein and thereof. In some embodiments, a phagocytosis stimulating agent may not be expressed by a cancer cell. In some cases, a phagocytosis stimulating agent can be exogenous to a cancer cell. In some instances, a phagocytosis stimulating agent can be specific to a cancer cell. In some instances, a phagocytosis stimulating agent may not be specific to a cancer cell.

In some instances, a phagocytosis stimulating agent can be linked to an attenuated cancer cell. In some instances, a phagocytosis stimulating agent can be attached to an attenuated cancer cell.

A phagocytosis stimulating agent can have an origin in viruses, gram-positive bacteria, gram-negative bacteria, fungi, protozoa, protists, nematodes, plant cells, animal cells, any derivatives herein and thereof, or any combinations herein and thereof. A phagocytosis stimulating agent can also be synthesized in vitro, such as but not limited to organic synthesis. A phagocytosis stimulating agent can be an intracellular, extracellular, lysosomal, endosomal, nuclear, cytoplasmic, mitochondrial, ER-bound, Golgi-bound, membrane-associated, or integrated membrane component. In some cases, a phagocytosis stimulating agent can also comprise triacyl lipopeptide, diacyl lipopeptide, lipoteichoic acid, lipoprotein, peptidoglycan, lipoarabinomannan, porin, envelope glycoprotein, GPI-mucin, phospholipomannan, zymosan, beta-glycan double-stranded (ds) RNA, double-stranded DNA, single-stranded (ss) RNA, single-stranded DNA, lipopolysaccharide, arabinogalactan, glycoinositolphospholipid, heat shock proteins (HSPs), flagellin, CpG DNA, methylated DNA, 5′-triphosphate RNA, diaminopimelic acid, triacyl lipopeptides, muramyl dipeptide (MDP), surface glycoprotein (GP), membrane components, lipoteichoic acid (LTA), phosphorylcholine (PC), PE, PI, mycolic acid, adenosine triphosphate (ATP), adenosine diphosphate (ADP) adenosine monophosphate (AMP), guanosine triphosphate (GTP), uridine triphosphate (UTP), thymidine triphosphate (TTP), cytidine triphosphate (CTP), GDP, UDP, TDP, CDP, GMP, UMP, CMP, uric acid crystals, phosphatidyinositol mannosides (PIM), endotoxin, wall teichoic acid (WTA), LTA, N-formylmethionine, carbohydrates, glucan, chitin, hamagglutinin, F-protein, phenol-soluble modulin, hemozoin, any derivatives herein and thereof, or any combinations herein and thereof. A dsDNA can be long or short. CpG DNA can comprise methylated or unmethylated CpG DNA. Arabinogalactan can comprise D-arabinose or D-galactose. A phagocytosis stimulating agent can comprise glucan or mannan.

As provided herein, a phagocytosis stimulating agent can comprise mannan or its derivatives. Mannan is often found on the yeast cell wall. It can comprise a series of mannose units linked by alpha (1-6) linkages. Mannan can also have alpha (1-2) and alpha (1-3) branched linkages. Detection of mannan leads to cell lysis or phagocytosis in the mannan-binding lectin (MBL) pathway. Mannans can also be found in plants, algae, fungus, or bacteria. They are synthesized from activated nucleotide sugars such as GDP-mannose, GDP-glucose, and UDP-galactose. Glycosyltransferases, localized in Golgi, utilize the activated nucleotide sugars to synthesize the polymer by facilitating the linkage between mannose monomers.

Mannan can comprise the polysaccharide moiety of glycoproteins. Mannan can comprise a linear, branched, or a linear and branched polymer of linked mannose resides or molecules. In some cases, mannan can have beta (1-4) linkages.

Mannan can be cytoplasmic or extracellular. Mannan can have a molecular weight of 666.6 g/mol. In some embodiments, mannan can have 14 hydrogen bond donors and 21 acceptors. In other cases, mannan can have 10 rotatable bonds. In some instances, mannan can have a monoisotopic mass of 666.221858 g/mol. In other cases, mannan can have a topological polar surface area of 348 Å². In some cases, mannan can have 45 heavy atoms and 0 formal charge. In some embodiments, mannan can be (2S,3 S,4S,5 S,6R)-2-[(2R,3 S,4R,5R,6S)-6-[(2R,3 S,4R,5 S,6S)-4,5-dihydroxy-2-(hydroxymethyl)-6-[(2R, 3R,4R,5 S,6R)-4,5,6-trihydroxy-2-(hydroxymethyl) oxan-3-yl]oxyoxan-3-yl] oxy-4,5-dihydroxy-2-(hydroxymethyl) oxan-3-yl] oxy-6-(hydroxymethyl)oxane-3,4,5-triol. In some cases, mannan can be C₂₄H₄₂O₂₁.

Manan can comprise a backbone of alpha (1-6) linked mannose unites with alpha (1-2) and alpha (1-3) linked side chains. The side chains can have 0, 1, 2, 3, 4, 5, 6, 7, 8, 9, 10, or more sugar units in length. In some embodiments, a side chain can comprise a mannan-oligosaccharide (MOS). A mannose can be a D-mannose or L-mannose. A mannose can have a molecular formula of C₆H₁₂O₆. In some embodiments, a mannose can have a molecular weight of 180.16 g/mol. In other cases, a mannose can comprise a D-manno-hexopyranose. A mannose can also be (3S,4S,5S,6R)-6-(hydroxymethyl)oxane-2,3,4,5-tetrol. A mannose can comprise two different-sized rings, a six-membered pyranose form and a five-membered furanose form. In some instances, a ring can have an alpha or beta configuration at the anomeric position.

Mannan can be synthesized by yeast. Mannan-producing yeasts can comprise Hansenula holstii, Rhodotorula acheniorum, Sporobolomyces salmonicolor, Saccharomyces cerevisiae, Candida albicans, Schizosaccharomyces pombe, Meyerozyma guilliermondii, Brewers dried yeast, or Candida utilis. Mannan can be synthesized by plants. Mannan-producing plants can comprise the Ebenaceae family, Arabidopsis thaliana, the Leguminoseae family, Caesalpinia spinosa Kuntze, the Annonaceae family, Amorphophallus konjac, Ceratonia siliqua, the Convolvulaceae family, Cyamopsis tetragonoloba, the Loganiaceae family, Senna tora, Trigonella foenum-graecum L., the Palmae family, Picea abies, Cercis siliquastrum, or Nicotiana plumbaginifolia. Other mannan-producing organisms can comprise Porphyra umbilicalis, Acetabularia acetabulum, Charophyceae, Dactylium dendroides, Pseudocyphellaria clathrata, Pseudomonas mutabilis, Pseudomonas syringae pv. ciccaronei, Edwardsiella tarda, Pseudomonas aeruginosa, or Brevibacillus thermoruber.

A phagocytosis stimulating agent can comprise a linker that link the phagocytosis stimulating agent to an attenuated cancer cell by a linker. A linker can comprise a chemical linkage, physical linkage, association, attachment, connection, junction, placement, fusion, interaction, ligation, chemical bond, physical bond, crosslink, joint, coupling, clamping, tie, or any derivatives herein and thereof, or any combinations herein and thereof. A linker can comprise a chemical or physical linker. A chemical linker can comprise a membrane linker. A membrane linker can comprise a myristate, palmitate, farnesyl, geranylgeranyl, oleate, isoprenoid, fatty acid, diacylglycerol, long-chain acyl group, long-chain prenyl group, cholesterol, stearyl, phosphatidyl-ethanolamine (PE), phosphatidylinositol (PI), glycosyl phosphatidyl inositol (GPI) anchor, chelator lipid anchor, polypeptide, derivatives herein and thereof, or any combinations herein and thereof. A chelator lipid anchor can comprise nitrilotriacetic acid ditetradecylamine (NTA-DTDA).

A linker can also comprise Biocompatible Anchors for Membrane (BAM). A BAM can comprise a lipid anchor. A lipid anchor can comprise any lipid anchors described herein and thereof. BAM can comprise a lipid anchor and a Polyethylene glycol (PEG) chain. BAM can be used to link a chemical, molecule, polypeptide, nucleic acid, lipid, carbohydrate, any moieties described herein and thereof, any derivatives herein and thereof, any combinations herein and thereof to a cell.

In some cases, BAM can comprise an NHS reactive ester group. In other cases, a PEG chain can be hydrophilic. In some embodiments, BAM can comprise a succinylated poly(ethylene glycol) oleyl ether at the hydroxyl end of a PEG chain. In other cases, BAM can comprise a N-hydroxysuccinimide (NHS) at the succinyl PEG end. In some cases, BAM comprising an NHS end can bind most proteins. In some cases, a BAM can comprise a Oleyl-O-poly(ethylene glycol)-succinyl-N-hydroxy-succinimidyl esters.

Normally, in the context of cancer immunology, the tumor microenvironment has been the subject of intense study for its proposed role in suppressing tumor-specific immune responses and promoting tumor progression. Indeed, efforts to interrupt activation of immunosuppressive checkpoint pathways have taken center stage in the field of immuno-oncology and have culminated in the development and introduction of immune checkpoint inhibitors (ICIs). Promising therapeutic outcomes have been observed with PD-1/PD-L1 and CTLA-4 inhibitors, promoting the growth of ICI-based clinical trials and paving the road for approval of ICIs in various cancer treatments. Initial success of anti-CTLA-4 and anti-PD-1/PD-L1 therapies, however, have been blunted in part by the low response rate associated with ICIs.

In this regard, emerging studies are demonstrating that tumors with high mutational burdens may play a role in affecting the extent of T-cell infiltration within the tumor microenvironment. The extent of T-cell infiltration within different tumors is increasingly being recognized as an important prognostic biomarker for response to ICI's as it suggests the presence of a pre-established immune response within the tumor microenvironment that can potentially be re-invigorated with appropriate immunotherapeutic combinations.

The immunotherapeutic strategy, introduced in this invention, leverages the use of burgeoning mRNA vaccine technology as well as both phagocytosis stimulating ligands and immunostimulatory adjuvants for directing an immune response against tumor-specific antigens (TSA).

Further reference is made to the following experimental examples.

### EXAMPLES

The following examples are given for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

mRNA Nanoparticle Vaccine Encoding AH1 Peptide Prevents Tumor Recurrence in CT26 Colon Carcinoma Model.

AH1 peptide is a well-established tumor specific antigen of CT26 colon carcinoma. An mRNA nanoparticle will be generated that encodes the AH1 peptide (mRNA-AH1 in brief). It will be determined if mRNA-AH1 treatment in wild type mice can help establish a tumor specific immune response against CT26 cell implantation. The mRNA-AH1 nanoparticle will be generated in the same way as previously reported. See Corbett et al., “SARS-CoV-2 mRNA vaccine design enabled by prototype pathogen preparedness,” Nature 586, 567-571 (2020); https://doi.org/10.1038/s41586-020-2622-0. mRNA-AH1 will then be administered to wild type mice as two intramuscular injections three weeks apart. mRNA-AH1 treatment will be alone or in combination with TLR agonists (LTA, poly(I:C), R-848) and anti-CD40 antibody. Three weeks after the second injection, peripheral blood will be collected to compare the immune response against CT26 cells in different groups.

Flow cytometry staining will be performed to the extent of dendritic cell maturation/activation, CD8-T cell activation, and tumor specific memory T cells. An ELISA assay will also be performed to evaluate the tumor-killing cytokines after coculturing the PBMC with CT26 tumor cells. Five weeks after the second injection, these mice will be inoculated with CT26 tumor cells subcutaneously. Untreated wild type mice will also be implanted with CT26 cells as a control. Tumor growth size in both control and treatment groups will be monitored every other day. Immune profiling will also be performed in the tumor, draining lymph node, and spleen after euthanizing these mice.

### Example 2

Administration of Irradiated Tumor Cells Expressing SARS-CoV-2 S (Spike) Protein Inhibits Tumor Growth in SARS-CoV-2 Vaccinated Mice.

To take advantage the herd immunity among a population, including cancer patients, one goal of this invention is a method to trigger an immune response by targeting expression of such a herd-immunized antigen in ex vivo cultured cancer cells. It will be determined if administration of irradiated tumor cells expressing SARS-CoV-2 S protein inhibits CT26 tumor cell growth in SARS-CoV-2 vaccinated mice. The SARS-CoV-2 vaccinated Balb/c mice will be established as previously reported. See Corbett et al. (2020).

CT26 cells will be infected by adenovirus that expresses SARS-CoV-2 S protein, so the infected CT26 cells will express SARS-CoV-2 S protein (CT26-S cell in brief). The tumor model will be established by subcutaneous injection of wild type CT26 cells in SARS-CoV-2 vaccinated mice. Once the mean tumor size reach 100 mm³, these tumor-bearing mice will be randomized into three groups: (1) saline control, (2) irradiated CT26-S cell only: CT26-S cells will be irradiated at 100 Gy and then injected subcutaneously every day for 3 days then repeated weekly for a total of 4 weeks, (3) irradiated CT26-S cell with adjuvants (LTA, poly I:C, R-848, and anti-CD40): irradiated CT26-S cells will be mixed with adjuvants before subcutaneous injection. The tumor size will be monitored every other day.

Comprehensive immune profiling analysis will also be performed to examine the effects on: (1) cell composition of innate immunity (dendritic cells, neutrophil, monocyte, macrophage, NK cells, etc) and adaptive immunity (CD4-T, CD8-T, T-reg, B cells), (2) T cell differentiation (CD62L, CD44) and exhaustion (TIM3, PD1, LAG3, CTLA4), (3) T cell function (IFN-gama, TNF-alpha, Granzyme B, etc), and (4) tumor specificity (MHC tetramer, co-culture, etc).

### Example 3

Administration of Irradiated Tumor Cells Expressing SARS-CoV-2 S (Spike) Protein Prevents Tumor Metastasis in Melanoma Mouse Model.

To take advantage of the herd immunity such as SARS-CoV-2, in general population, including cancer patients, one goal of this invention is to trigger an immune response in ex vivo cultured cancer cells by expressing of the herd-immunized antigen. The experiment will be performed by the administration of irradiated tumor cells expressing SARS-CoV-2 S protein in SARS-CoV-2 vaccinated mice first and then followed up with the inhibition of B16-F10 tumor cell metastasis. The SARS-CoV-2 vaccinated C57BL/6J mice will be established as previously reported. See Corbett et al. (2020).

Expression of SARS-CoV-2 S protein in B16-F10 cells (B16-S cell in brief) will be achieved by lentivirus or adenovirus mediated gene delivery system. To examine if B16-S cells can trigger the innate and adaptive immune response against B16-F10 melanoma cells, SARS-CoV-2 vaccinated C57BL/6J mice will be treated in four groups: (1) saline control, (2) irradiated B16-S cell only, (3) irradiated B16-S cell mixed with adjuvants (LTA, poly I:C, R-848, and anti-CD40), (4) irradiated naïve B16-F10 cell with adjuvants (LTA, poly I:C, R-848, and anti-CD40). For each treatment, the indicated cells will be irradiated at 100 Gy. The components in each group will be injected subcutaneously every day for 3 days then repeated weekly for a total of 4 weeks. After the complete treatment cycle, naive B16-F10 tumor cells will be injected through tail vein in all four groups of mice. Melanoma metastatic loci in lungs will be compared among four groups after two or three weeks.

Histologic examinations will be performed on lung tissues to detect the metastasis loci in all four groups of mice. Splenocytes will be isolated and co-cultured with B16-F10 tumor cells to confirm if there is tumor-specific immune response in the treated mice. Supernatant from the co-culture media will also be used for ELISA test to evaluate the tumor-killing cytokines.

As will be appreciated from the descriptions herein, a wide variety of aspects and embodiments are contemplated by the present disclosure, examples of which include, without limitation, the aspects and embodiments listed below:

A personalized cancer vaccine containing attenuated cancer cells that express an antigenic polypeptide where the antigenic polypeptide is membrane anchored, and the personalized tumor vaccine, when administered to a subject in need thereof, is effective to activate an immune response;

A personalized cancer vaccine, where the expression of an antigenic polypeptide can be achieved through, for example, mRNA electroporation or an adenovirus vector;

A personalized cancer vaccine described herein, where the attenuated cancer cells can be prepared by the following steps: (1) harvesting cancer cells from a biopsy of a site of tumor from the individual, (2) culturing the harvested cancer cells to a therapeutically relevant amount, and (3) irradiating the cultured cancer cells;

A personalized cancer vaccine described herein, where the antigenic polypeptide of the is derived from a protein from a strain of Influenza A virus or an immunogenic fragment thereof, further where examples of the protein from a strain of Influenza A virus could include influenza hemagglutinin 1 (HA1), HA2, HA7, HA10, neuraminidase protein, or a combination thereof;

A personalized cancer vaccine described herein, where the antigenic polypeptide could be a herpes simplex virus antigenic polypeptide or an immunogenic fragment thereof;

A personalized cancer vaccine described herein, where the antigenic polypeptide is derived from a protein from human coronavirus SARS-CoV-2 or an immunogenic fragment thereof, further where examples of the protein from SARS-CoV-2 includes Coronavirus (CoV) S1 protein (spike protein), an envelope protein, a membrane protein, a nucleocapsid protein, or a combination thereof;

A personalized cancer vaccine described herein that further contains a phagocytosis stimulating agent and an immunostimulatory adjuvant, where the phagocytosis stimulating agent could be a mannan, and the immunostimulatory adjuvant could be a Toll like 80 receptor (TLR) agonist, an anti-CD40 antibody, or a combination thereof, further where examples of the TLR agonists include LTA, Poly I:C, and R-848, and the anti-CD40 antibodies can be agonistic;

A cancer vaccine that includes a first agent comprising at least one ribonucleic acid (RNA) polynucleotide(s); and a second agent comprising a phagocytosis stimulating agent and an immunostimulatory adjuvant;

A cancer vaccine as described herein that is formulated for delivery to a tumor organ where upon being delivered to the tumor organ, the first agent is configured to produce a membrane anchored antigenic polypeptide therein and trigger an immune response; and the second agent is effective to amplify the immune response;

A cancer vaccine as described herein where the delivery of the cancer vaccine is through an intratumoral injection;

A cancer vaccine as described herein where the membrane anchored antigenic polypeptide of the cancer vaccine can be derived from a strain of Influenza A virus, a herpes simplex virus, or human coronavirus SARS-CoV-2;

A cancer vaccine as described herein where the first agent of the cancer vaccine is formulated in a lipid nanoparticle composition;

A cancer vaccine as described herein where the second agent, the phagocytosis stimulating agent, again, is a mannan;

A cancer vaccine as described herein where the immunostimulatory adjuvant is a Toll like receptor (TLR) agonist, an anti-CD40 antibody, or a combination thereof.

A personalized cancer vaccine that includes a first agent of comprising at least one ribonucleic acid (RNA) polynucleotide and a second agent comprising a phagocytosis-stimulating agent and an immunostimulatory adjuvant;

A personalized cancer vaccine as described herein that is formulated to be administered to a cancer patient in need thereof and the RNA polynucleotide encodes at least one neoantigen derived from said cancer patient;

A personalized cancer vaccine as described herein where upon administration to the cancer patient, the first agent is configured to produce an antigenic polypeptide therein and trigger an immune response; and the second agent is effective to amplify the immune response;

A personalized cancer vaccine as described herein where the RNA polynucleotide can contain at least an open reading frame of one tandem minigene construct comprising a sequence encoding at least one neoantigen derived from said cancer patient;

A personalized cancer vaccine as described herein where the first agent can be formulated in a nanoparticle composition, the phagocytosis stimulating agent can be a mannan, the immunostimulatory adjuvant can be a Toll like receptor (TLR) agonist, an anti-CD40 antibody, or a combination thereof;

Any of the vaccines as described herein where the vaccine is administered to a subject as a method for treating a cancer by administering to a subject having the cancer an effective amount of any vaccine as described herein;

Any of the vaccines as described herein where the vaccine is administered by a subcutaneous inoculation;

Any of the vaccines as described herein where the vaccine is administered to a mammal, e.g., a human or a mouse.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. To the extent a definition of a term set out in a document incorporated herein by reference conflicts with the definition of a term explicitly defined herein, the definition set out herein controls.

1. A personalized cancer vaccine comprising attenuated cancer cells that express an antigenic polypeptide, wherein the antigenic polypeptide is membrane anchored and the personalized tumor vaccine, when administered to a subject in need thereof, is effective to activate an immune response.

2. The personalized cancer vaccine of claim 1, wherein the antigenic polypeptide is derived from a protein from a strain of Influenza A virus or an immunogenic fragment thereof.

3. The personalized cancer vaccine of claim 2, wherein the protein from a strain of Influenza A virus comprises influenza hemagglutinin 1 (HA1), HA2, HA7, HA10, neuraminidase protein, or a combination thereof.

4. The personalized cancer vaccine of claim 1, wherein the antigenic polypeptide comprises a herpes simplex virus antigenic polypeptide or an immunogenic fragment thereof.

5. The personalized cancer vaccine of claim 1, wherein the antigenic polypeptide is derived from a protein from human coronavirus SARS-CoV-2 or an immunogenic fragment thereof.

6. The personalized cancer vaccine of claim 5 wherein the protein from SARS-CoV-2 comprises Coronavirus (CoV) S1 protein (spike protein), an envelope protein, a membrane protein, a nucleocapsid protein, or a combination thereof.

7. The personalized cancer vaccine of claim 1, wherein the attenuated cancer cells are prepared by i) harvesting cancer cells from a biopsy of a site of tumor from the individual, ii) culturing the harvested cancer cells to a therapeutically relevant amount, and then iii) irradiating the cultured cancer cells.

8. The personalized cancer vaccine of claim 1, further comprising a phagocytosis stimulating agent and an immunostimulatory adjuvant.

9. The personalized cancer vaccine of claim 8, wherein the phagocytosis stimulating agent comprises a mannan.

10. The personalized cancer vaccine of claim 8, wherein the immunostimulatory adjuvant comprises a Toll like receptor (TLR) agonist, an anti-CD40 antibody, or a combination thereof.

11. A cancer vaccine comprising: a first agent comprising at least one ribonucleic acid (RNA) polynucleotides; and a second agent comprising a phagocytosis stimulating agent and an immunostimulatory adjuvant, wherein:
the cancer vaccine is formulated for delivery to a tumor organ;
upon being delivered to the tumor organ, the first agent is configured to produce a membrane anchored antigenic polypeptide therein, and
trigger an immune response;
and, wherein the second agent is effective to amplify the immune response.

12. The cancer vaccine of claim 11, wherein the membrane anchored antigenic polypeptide is derived from a protein from a strain of Influenza A virus or an immunogenic fragment thereof.

13. The cancer vaccine of claim 12, wherein the protein from a strain of Influenza A virus comprises influenza hemagglutinin 1 (HA1), HA2, HA7, HA10, neuraminidase protein, or a combination thereof.

14. The cancer vaccine of claim 11, wherein the membrane anchored antigenic polypeptide comprises a herpes simplex virus antigenic polypeptide or an immunogenic fragment thereof.

15. The cancer vaccine of claim 11, wherein the membrane anchored antigenic polypeptide is derived from a protein from human coronavirus SARS-CoV-2 or an immunogenic fragment thereof.

16. The cancer vaccine of claim 15, wherein the protein from SARS-CoV-2 comprises Coronavirus (CoV) S1 protein (spike protein), an envelope protein, a membrane protein, a nucleocapsid protein, or a combination thereof.

17. The cancer vaccine of claim 11, wherein the first agent is formulated in a lipid nanoparticle composition.

18. The cancer vaccine of claim 11, wherein the phagocytosis stimulating agent comprises a mannan.

19. The cancer vaccine of claim 11, wherein the immunostimulatory adjuvant comprises a Toll like receptor (TLR) agonist, an anti-CD40 antibody, or a combination thereof.

20. A personalized cancer vaccine comprising:
a first agent of comprising at least one ribonucleic acid (RNA) polynucleotide; and
a second agent comprising a phagocytosis stimulating agent and an immunostimulatory adjuvant,
wherein the personalized cancer vaccine is formulated to be administered to a cancer patient in need thereof.

21. The personalized cancer vaccine of claim 20, wherein:
upon administration to the cancer patient, the first agent is configured to produce an antigenic polypeptide therein and trigger an immune response;
the RNA polynucleotide encodes at least one neoantigen derived from said cancer patient; and the second agent is effective to amplify the immune response.

22. The personalized cancer vaccine of claim 20, wherein the RNA polynucleotide comprises at least an open reading frame of one tandem minigene construct comprising a sequence encoding at least one neoantigen derived from said cancer patient.

23. The personalized cancer vaccine of claim 20, wherein the first agent is formulated in a nanoparticle composition.

24. The personalized cancer vaccine of claim 20, wherein the phagocytosis stimulating agent comprises a mannan.

25. The personalized cancer vaccine of claim 20, wherein the immunostimulatory adjuvant comprises a Toll like receptor (TLR) agonist.

26. The personalized cancer vaccine of claim 20, wherein the immunostimulatory adjuvant comprises an anti-CD40 antibody.

27. A method for treating a cancer, the method comprising administering to a subject having the cancer an effective amount of a personalized cancer vaccine of claim 1.

28. The method of claim 27, wherein the personalized cancer vaccine is administered by a subcutaneous inoculation.

29. A method for treating a cancer, the method comprising administering to a subject having the cancer an effective amount of a cancer vaccine of claim 11.

30. The method of claim 29, wherein the cancer vaccine is administered by intratumoral injection.

31. A method for inducing an immunological memory against a cancer, the method comprising administering to a subject having the cancer an effective amount of a personalized cancer vaccine of claim 20.

32. The method of claim 31, wherein the personalized cancer vaccine is administered by a subcutaneous inoculation.
